(19) United States
(12) Patent Application Publication
(10) Pub. No.: US 2025/0049379 A1
(43) Pub. Date: Feb. 13, 2025

(54) MYOTRACE CONTINUOUS MONITORING SCENARIOS

(71) Applicant: KONINKLIJKE PHILIPS N.V., EINDHOVEN (NL)

(72) Inventors: MARIAN DEKKER, Noord Brabant (NL); JENS MUHLSTEFF, AACHEN (DE)

(21) Appl. No.: 18/720,810

(22) PCT Filed: Dec. 5, 2022
(86) PCT No.: PCT/EP2022/084331

§ 371 Date: Jun. 17, 2024

Related U.S. Application Data
(60) Provisional application No. 63/290,875, filed on Dec. 17, 2021.

(51) Int. Cl. A61B 5/389; A61B 5/00; A61B 5/113; A61B 5/257

(52) U.S. Cl. CPC A61B 5/389; A61B 5/113; A61B 5/257; A61B 5/6823; A61B 5/6833; A61B 5/742; A61B 2562/0219

(57) ABSTRACT

A non-invasive system for monitoring neural respiratory drive (NRD) in addition to other patient metrics typically monitored non-invasively utilizes continuous parasternal EMG signal measurements and can be integrated into early warning scoring systems (EWS) used to perform spot checks of patients in general hospital wards. Using the assessment of a pulmonologist as the gold standard for determination of respiratory status, the disclosed system assesses a patient's respiratory status significantly more accurately than EWS systems that only use parameters such as respiration rate, since NRD determinations provide an objective quantification of the effort that it takes for a patient to breathe. In particular, the NRD determination system takes EMG measurements of the upper chest inspiratory muscles during either or both regular breathing inhalation and maximum effort sniff activity, as EMG measurements taken during inhalation are considered to be indicators of the balance between respiratory muscle load and respiratory muscle capacity.

### BACKGROUND OF THE INVENTION

### 1. Field of the Invention

The disclosed concept pertains to methods and systems for continuous monitoring of the respiratory condition of patients and, in particular, to methods and systems for improving wearable devices for continuous, non-invasive monitoring of respiratory status and other metrics of patients.

### 2. Description of the Related Art

Early warning scoring (EWS) systems for detecting deterioration and tracking trends in patient health status are often used in lower acuity healthcare settings, such as a general ward of a hospital. FIG. 1 shows a diagnostic chart 1 listing several example parameters that may be monitored in a typical implementation of an EWS system. As shown in FIG. 1, heart rate, systolic blood pressure, respiration rate, SpO₂, temperature, and level of consciousness are non-limiting representative examples of non-invasive physiological parameters that are often used to monitor the condition of patients in a hospital general ward. These parameters can be continuously monitored via the use of wearable patches structured to be affixed to the skin of the patient, via, for example, an adhesive backing. While factors other than those listed in diagnostic chart 1 may be monitored in a EWS system, the shown in FIG. 1 are representative of the combination of factors typically used to diagnose and monitor the general condition of patients in a general ward. Some factors listed in the diagnostic chart I may be weighed differently than others. For example, if a somewhat low temperature, somewhat low SPO2, or somewhat low respiration rate is detected, the patient may just need to be monitored without immediate drastic action being taken, but if systolic blood pressure is too low, more immediate and pronounced action may be taken.

With respect to adverse respiratory conditions, accurate detection of patient respiratory effort in a general ward is important, as 1 in 6 general ward patients is at 3 times higher risk of presenting a respiratory adverse event than if they were not in the general ward. Respiration rate is often monitored in EWS systems, since respiration rate can easily be measured non-invasively. However, although non-invasive measurement of respiration rate is informative, respiration rate alone does not provide a comprehensive picture of respiratory health, as it does not provide insight into the effort exerted by a patient while breathing. For example, if a relatively healthy person and a person with Chronic Pulmonary Obstructive Disease (COPD) were to exhibit the same respiration rate, the person with COPD exert significantly more effort to breathe at the same rate as the healthy person, and the respiration rate alone would not provide insight into how much effort each person was exerting to breathe at that rate.

Accordingly, there is room for improvement in methods and systems used to non-invasively monitor the respiratory condition of patients in a general hospital ward, in conjunction with other patient metrics typically measured non-invasively.

### SUMMARY OF THE INVENTION

Accordingly, it is an object of the present invention to provide non-invasive systems and methods for objectively quantifying the effort exerted by a patient while breathing, referred to as neural respiratory drive (NRD), in combination with other metrics typically measured non-invasively. The NRD measurement-capable monitoring systems and methods disclosed herein implement parasternal EMG signal monitoring that can be integrated into early warning scoring (EWS) systems used to perform spot checks of patient health status in general wards of hospitals. In particular, the NRD determination system takes EMG measurements of the upper chest inspiratory muscles during either or both regular breathing inhalation and maximum effort sniff activity, as EMG measurements taken during inhalation are considered to be indicators of the balance between respiratory muscle load and respiratory muscle capacity.

In one embodiment, a non-invasive patient monitoring system for non-invasively monitoring a respiratory status of a patient and at least one other non-respiratory metric of the patient comprises a sensor patch that includes a plurality of EMG electrodes structured to be affixed to an upper chest region of the patient and a number of non-EMG sensors, a controller electrically connected to the EMG electrodes, and a user interface in electrical communication with the controller. The controller is configured to determine a neural respiratory drive index based on EMG signal data sensed by the EMG electrodes during at least one of regular breathing activity and sniff activity.

In another embodiment, a method for non-invasively monitoring neural respiratory drive and other non-respiratory metrics of a patient includes positioning a sensor patch on an upper chest region of the patient, the sensor patch comprising a plurality of EMG electrodes and a number of non-EMG sensors: electrically connecting the sensor patch to a controller: electrically connecting the controller to a user interface: sensing EMG signal data with the plurality of EMG electrodes during inhalation activity performed by the patient; and determining a neural respiratory drive index with the controller based on a number of attributes of the inhalation activity. The inhalation activity comprises at least one of regular breathing or sniff activity. Electrically connecting the sensor patch (12) to the controller electrically connects the plurality of EMG electrodes and number of non-EMG sensors to the controller.

In another embodiment, a non-invasive patient monitoring system for non-invasively monitoring a respiratory status of a patient and at least one other non-respiratory metric of the patient comprises a sensor patch structured to be affixed to skin on an upper chest region of the patient and a user interface. The sensor patch comprises a plurality of EMG electrodes, a number of non-EMG sensors, and a controller electrically connected to the sensor patch. The user interface is in electrical communication with the controller, and the controller is configured to determine a neural respiratory drive index based on EMG signal data sensed by the EMG electrodes during at least one of regular breathing activity and sniff activity.

These and other objects, features, and characteristics of the present invention, as well as the methods of operation and functions of the related elements of structure and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a diagnostic chart listing parameters typically included in early warning scoring (EWS) systems used to monitor patient status in lower acuity patient settings;

FIG. 2 shows a system for non-invasively determining neural respiratory drive (NRD) of a patient in combination with other metrics typically measured non-invasively, in accordance with exemplary embodiments of the present invention:

FIG. 3 is flow chart of a method for non-invasively monitoring NRD of a patient, in accordance with exemplary embodiments of the present invention:

FIG. 4 is a Receiver Operating Characteristic (ROC) curve depicting the accuracy of patient respiratory status determinations made by a controller shown in FIG. 2, in accordance with exemplary embodiments of the present invention; and

FIG. 5 is a Receiver Operating Characteristic (ROC) curve depicting the accuracy of patient respiratory status determinations made by a controller using parameters typically included in early warning systems in lower acuity patient settings, such as the parameters included in the diagnostic chart shown in FIG. 1.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, the term “controller” shall mean a number of programmable analog and/or digital devices (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

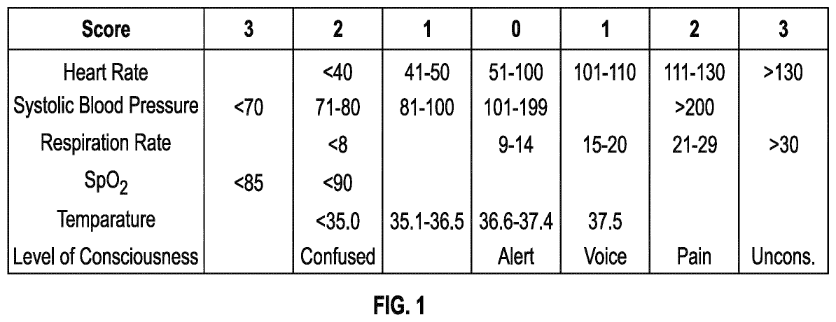

The present invention, as described in greater detail herein in connection with various particular exemplary embodiments, provides methods and systems for determining the breathing effort of a patient in a non-invasive manner, in combination with other metrics typically measured non-invasively. The disclosed methods and accurately determine if the patient is experiencing adverse respiratory conditions, and specifically determine the neural respiratory drive (NRD) of the patient in addition to the vital signs typically measured as part of early warning scoring (EWS) systems used for both spot checks and continuous monitoring of patients' health statuses in the general ward of a hospital. NRD provides a more accurate depiction of a patient's respiratory status than other non-invasive parameters that may be used to determine a patient's respiratory status, such as respiration rate alone or respiration rate considered in combination with other non-invasive measurements, such as those listed in diagnostic chart 1 shown in FIG. 1. In contrast with respiration rate, which does not provide an indication of the effort exerted by a patient to breathe at a given rate, NRD provides an objective measurement of breathing effort. It should be noted that methods and systems for determining NRD invasively are known, but the methods and systems disclosed herein are non-invasive and utilize skin-worn EMG sensors. In particular, the methods and systems disclosed herein determine NRD using EMG measurements of the upper chest inspiratory muscles, as EMG measurements taken during inhalation are considered indicative of the balance between respiratory muscle load and respiratory muscle capacity.

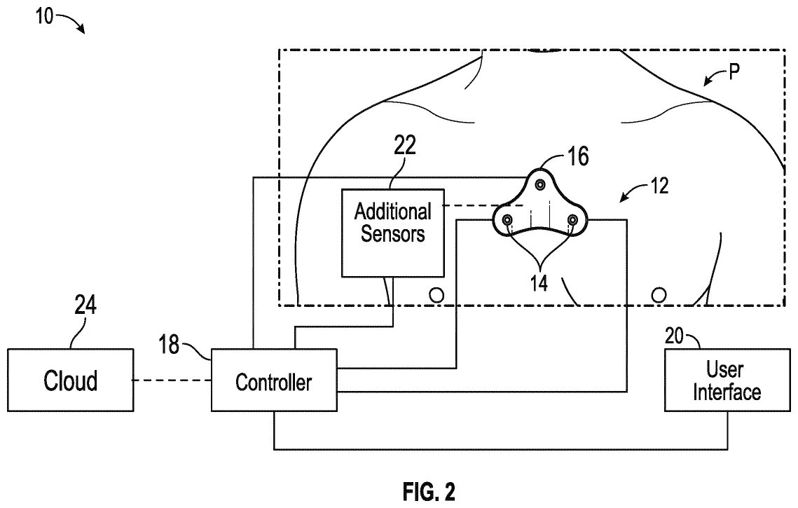

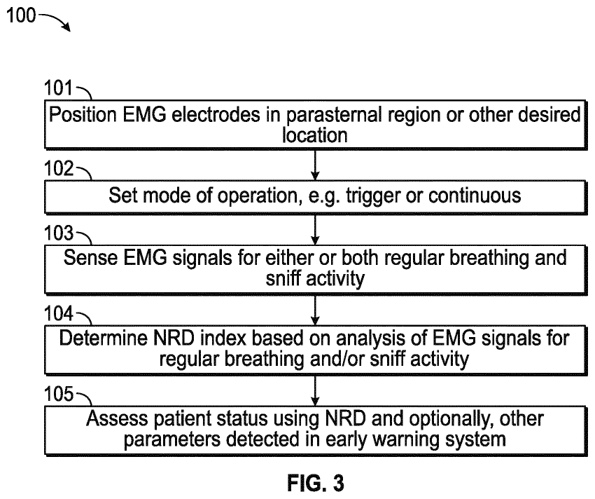

Referring now to FIG. 2, a non-invasive patient monitoring system 10 with NRD monitoring capability according to an exemplary embodiment of the present invention is shown. Monitoring system 10 comprises an Sensor patch 12 comprising a number of EMG signal electrodes 14 and a reference electrode 16, a controller 18, and a user interface 20. Sensor patch 12 is structured to be affixed to the skin of patient P via, for example and without limitation, an adhesive surface. In an exemplary embodiment, Sensor patch 12 is positioned such that signal electrodes 14 are positioned on the second intercostal space and reference electrode 16 is placed on the sternum slightly above (relative to the view shown in FIG. 2) reference electrodes 14 in order to optimally monitor respiratory muscle activity of patient P. However, it will be appreciated that placement of signal electrodes 14 and reference electrode 16 in other locations within the upper chest region may be desired and implemented without departing from the scope of the disclosed concept. The EMG signals sensed by electrodes 14,16 are subsequently used to determine NRD index, as disclosed later herein with respect to method 100 depicted in FIG. 3.

Controller 18 is configured to be in electrical communication with the electrodes of Sensor patch 12 and user interface 20. This enables controller 18 to receive and store the signals measured by the EMG electrodes 14,16, and enables a clinician, caregiver, or even patient P to receive the results of any processing performed by controller 18 (as detailed further herein) via user interface 20, as well as to input commands to controller 18. Sensor patch 12 is configured for monitoring a combination of metrics non-invasively, i.e. NRD and at least one other metric, wherein the at least one other metric can comprise, for example and without limitation, one of the metrics listed in the “Score” column in FIG. 1. Accordingly, sensor patch 12 includes additional sensors 22 beyond the EMG electrodes 14,16. Additional sensors 22 can comprise, for example and without limitation, an accelerometer, a chest SpO₂ sensor, or a core temperature sensor. Exemplary uses of these optional sensors 22 are detailed further later herein with respect to detailing how NRD monitoring system 10 can be operated in various continuous and trigger modes of operation. While the depiction of additional sensors 22 in FIG. 2 may convey the impression that additional sensors 22 are external to sensor patch 12, it should be noted that the depiction of additional sensors 22 in FIG. 2 is for the purpose of clearly establishing that sensor patch 12 includes additional sensors beyond electrodes 14,16, and that additional sensors 22 are integrated within sensor patch 12. While FIG. 2 depicts connections between the EMG electrodes 14,16 and controller 18, as well as connections between controller 18 and user interface 20, it should be noted that the connections shown are solely intended to depict electrical communication between electrodes 14,16 and controller 18, and between controller 18 and user interface 20, and that said communication can be facilitated via cither wired or wireless communication means, without departing from the scope of the disclosed concept. In some exemplary embodiments, Sensor patch 12 can be a passive patch such that measurements can only be taken when external power is provided to the patch, and in other exemplary embodiments, Sensor patch 12 can be an active patch that includes an onboard power supply and processing means. In one non-limiting exemplary implementation of Sensor patch 12 as an active patch, controller 18 is integrated into Sensor patch 12 such that the aforementioned onboard power supply and processing means are provided by controller 18.

Regarding communication between controller 18 and user interface 20, while controller 18 and user interface 20 are depicted as two separate entities in FIG. 2, it should be noted that in some exemplary embodiments user interface 20 may be integrated into the same physical structure as controller 18, while in other exemplary embodiments controller 18 and user interface 20 exist in separate structures. For example and without limitation, in a hospital setting, user interface 20 can comprise a patient monitor, while in an embodiment of NRD monitoring system 10 implemented for at-home use, user interface 20 can comprise a personal mobile phone of the patient. It will be appreciated that controller 18, whether implemented on-board to Sensor patch 12 or externally to Sensor patch 12, can optionally be configured to link the data sensed by Sensor patch 12 to a computing cloud 24.

Referring now to FIG. 3, a flow chart of a method 100 for non-invasively monitoring NRD of a patient is shown, in accordance with an exemplary embodiment of the disclosed concept. The method of FIG. 3 can be executed, for example, by NRD monitoring system 10 shown in FIG. 2, and the method is described in conjunction with NRD monitoring system 10. However, it will be appreciated that the method may be employed in other devices as well without departing from the scope of the disclosed concept.

At step 101, sensor patch 12 is positioned on patient P as shown in FIG. 2 or as otherwise desired in order to measure respiratory muscle activity of patient P. When an implementation of NRD monitoring system 10 is being used wherein controller 18 is not implemented on-board to sensor patch 12, wires are connected between sensor patch 12 and controller 18 during step 101 as well. It will be appreciated that controller 18 is configured to be in electrical communication with user interface 20 without additional setup from a user. It will also be appreciated that the skin of patient P may need to be prepared in order to optimize EMG signal sensing. For example, the skin may need to be cleaned and/or shaved.

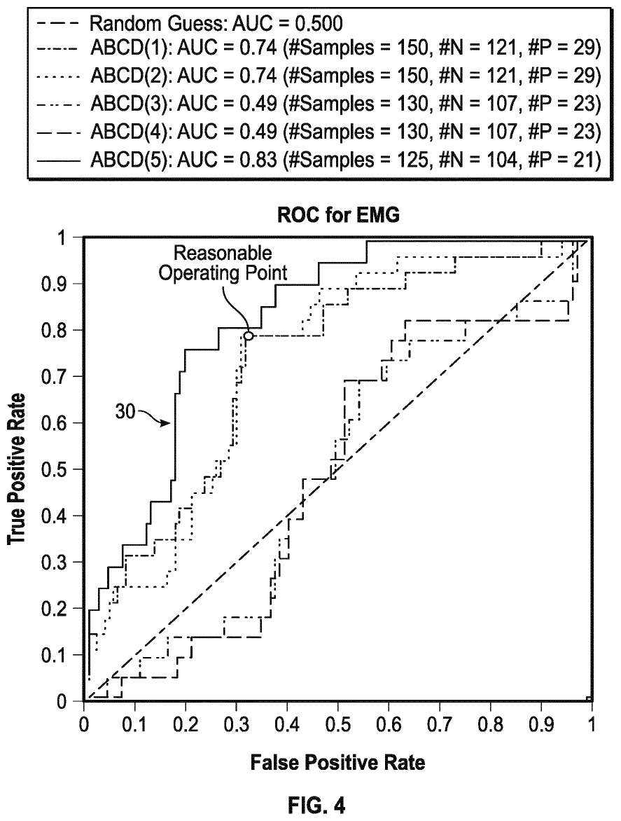

At step 102, the mode of operation of controller 18 is selected. As detailed further later herein with respect to FIG. 4, in an exemplary embodiment, controller 18 can be operated in either a trigger mode or a continuous mode. At step 103, EMG signals produced by patient P during either of or both regular breathing and sniff activity are sensed by sensor patch 12. The decision of whether only regular breathing or sniff activity or both is used to determine NRD is detailed further herein with respect to FIG. 4. In an exemplary embodiment of the disclosed concept, patient P performs onc minute of regular breathing followed by one minute of sniffing interspersed with regular breathing, with a sniff being defined as a deep, sharp inhalation that is perceived by patient P to require maximum inhalation effort.

At step 104, a NRD index is determined based on a number of attributes of the regular breathing and/or sniff EMG signals detected at step 102. The NRD index is a quantification of NRD based on various attributes of EMG signals recorded during either or both regular breathing and sniff activity performed by patient P. The possible attributes of the regular breathing and sniff EMG signals that can be used to determine the NRD index are detailed further herein with respect to FIG. 4. At step 105, the status of patient P is determined based upon the calculated NRD index, and if desired, the other parameters included in the EWS system are also taken into consideration. For example and without limitation, the respiratory status of patient P can be determined based upon the determined NRD index alone, while the overall status of the patient may be determined using all factors in the EWS system or only a subset of factors in the EWS system 1, depending on the reason for monitoring the patient.

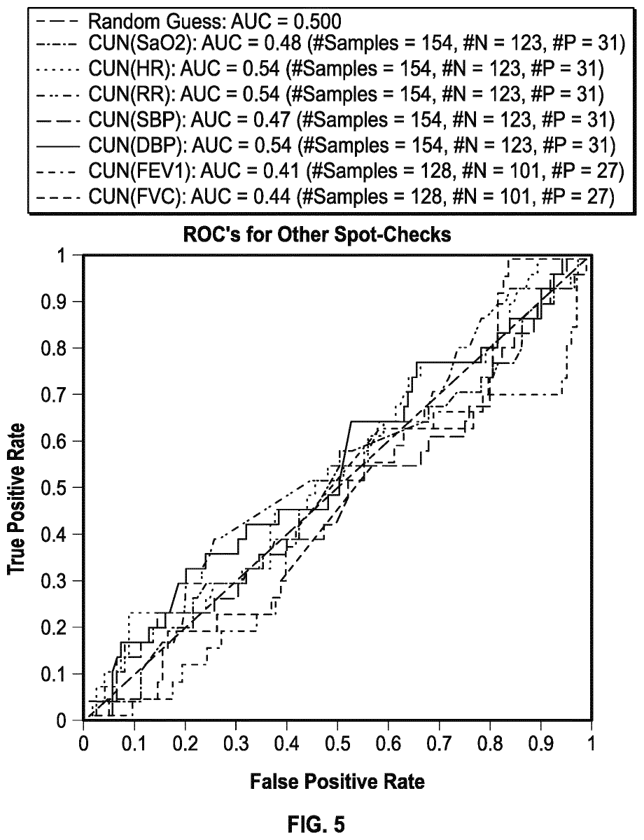

Referring now to FIGS. 4 and 5, two different sets of Receiver Operating Characteristic (ROC) curves are shown. FIG. 4 shows a set of ROC curves representing respiratory status determinations made by a controller that calculates NRD index such as controller 18 in NRD monitoring system 10 (FIG. 2), while FIG. 5 shows a set of ROC curves representing respiratory status determinations made by a controller using the traditional EWS system parameters shown in diagnostic chart 1 (FIG. 1). For the data shown in both FIG. 4 and FIG. 5, a pulmonologist's determination of a patient's respiratory status was used as the gold standard to which the controller's determination was compared, and the sensitivity and specificity for the data included in the curves was determined by comparing the determination made by the controller to the determination made by the pulmonologist.

In the figure legends for FIGS. 4 and 5, the term #Samples refers to the number of data points used to generate each ROC curve, i.e. the number of times that a patient's respiratory status was determined. For example, if an ROC curve was generated based on data collected for I patient whose respiratory status was checked 2 times a day for 10 days, then the number of samples would equal 20, whereas if an ROC curve was generated based on data collected for 5 patients whose respiratory status was checked 2 times a day for 15 days, then the number of samples would equal 150. The term #N indicates the number of times that a patient's respiratory status had deteriorated relative to the status check immediately preceding the current status check, and the term #P indicates the number of times that a patient's respiratory status had improved relative to the status check immediately preceding the current status check. The data expressed in the ROC curves shown in FIGS. 4 and 5 represents the number of times that a controller correctly determined (correctness being relative to a pulmonologist's assessment) whether or not a patient's respiratory status had deteriorated or improved since the previous status check. It will be appreciated that determinations of deterioration and improvement can be used, for example and without limitation, to evaluate whether a patient is responding well to a course of therapy/treatment or whether the patient is ready for discharge.

It is readily apparent from viewing FIGS. 4 and 5 that the reasonable operating points of the curves shown in FIG. 4 (one of which is labeled in FIG. 4) are overwhelmingly distributed closer to the upper left-hand corner of the graph as compared to the curves shown in FIG. 5, therefore indicating that the NRD determinations made by system 10 in FIG. 2 have a significantly higher rate of sensitivity and specificity relative to a respiratory status determination made using the parameters shown in diagnostic chart 1 in FIG. 1. Specifically, when area under the curve (denoted as AUC in the figure legends of FIG. 4 and FIG. 5) of all of the ROC curves are considered, the curves in FIG. 4 exhibit significantly higher overall AUC values, as the ROC curves in FIG. 5 with the highest AUC have an AUC of 0.54, while the ROC curves in FIG. 4 with the highest AUC exhibit significantly greater AUC values ranging from 0.74 to 0.83. It will be appreciated that a pulmonologist, in thoroughly evaluating a respiratory patient, may order and review x-rays of the patient's respiratory system, measure FEV1, and/or take the patient history into account when determining the respiratory status of the patient, so it is notable that an automated, non-invasive method and system for evaluating respiratory status can produce an ROC curve with an AUC of 0.83 for a sample set of 125 (i.e. the methodology that resulted in the data used to plot ROC curve 30 in FIG. 4).

In FIG. 4, each ROC curve corresponds to a different set of attributes of regular breathing and/or sniff activity EMG signals being used for calculating the NRD index relative to the other ROC curves. In FIG. 5, the differences in the ROC curves represent the differences in the weight afforded to each factor included in diagnostic chart 1 of FIG. 1. Curve 30 in FIG. 4 represents the most accurate method for calculating the NRD index, since the data points in curve 30 demonstrate the overall highest sensitivity and specificity with an AUC of 0.83. Curve 30 reflects calculation of the NRD index by recording the EMG signal for regular breathing by the patient for 1 minute, recording EMG for sniffs interspersed with regular breathing by the patient for 1 minute (as previously stated with respect to Method 100 in FIG. 3, a sniff is defined as a deep, sharp inhalation that is perceived by patient P to require maximum inhalation effort), finding the maximum signal value from the peaks in inhalation portions of the regular breathing EMG signal, finding the maximum signal value in the sniff EMG signal, and finding the ratio of the maximum regular breathing EMG signal value to the maximum sniff EMG signal value.

As used hereinafter, the term “relative”, as it relates to determination of NRD index, indicates that attributes of both a regular breathing EMG signal and a sniff EMG signal are used to calculate the NRD index. Accordingly, as used hereinafter, the term “absolute”, as it relates to determination of NRD index, indicates that attributes of only one of a regular breathing EMG signal or a sniff EMG signal, but not both, are used to calculate the NRD index. It should be noted that in most cases where an absolute NRD index is calculated, the attributes of a regular breathing EMG signal are used rather than the attributes of a sniff EMG signal, but this is not a requirement.

Non-limiting examples of methods used to calculate the NRD index for the ROC curves shown in FIG. 4 other than curve 30 include evaluating only the maximum values of the peaks in a sniff EMG signal and evaluating only the duration of the inhalation peaks in a regular breathing EMG signal. It should be noted that, while relative NRD indexes calculated by comparing regular breathing and sniff activity have been found to more closely align with a pulmonologist's opinion of respiratory status deterioration or improvement more often than absolute NRD measurements based only on regular breathing activity or only on sniff activity, there may be situations where absolute measurements of NRD based only on regular breathing (or only on sniff activity) are preferable. For example, because sniffs are maximum effort maneuvers, a clinician may need to guide or motivate a patient to perform a good sniff, and severely ill patients are often unable to perform sniffs. In addition, sniffs are difficult to perform reproducibly.

Continuing to refer to FIG. 4, considering both the increased accuracy of relative NRD determinations that use both regular breathing and sniff EMG data, as well as some of the aforementioned limitations presented by using sniff EMG data to determine NRD, it will be appreciated that relative NRD determinations may be preferable in some contexts while absolute determinations using only regular breathing data (or only sniff data) may be preferable in other contexts. Accordingly, it should be noted that an exemplary embodiment of the NRD monitoring system 10 can be operated in at least two modes, wherein at least one mode is a trigger mode and another mode is a continuous mode, as previously stated with respect to step 102 of method 100 (FIG. 3). The trigger and continuous modes are detailed further herein below, but as a preliminary matter, it should be noted that trigger mode calculations of NRD index generally produce a relative NRD index based on both regular breathing and sniff activity EMG signal data, while, continuous mode calculations of NRD index generally produce an absolute NRD index based on only regular breathing EMG signal data.

In trigger mode, controller 18 only calculates a NRD index for patient P when a trigger condition occurs. Three non-limiting examples of triggers that can prompt controller 18 to calculate the NRD index include: (1) patient condition, (2) time elapsed since previous measurement, and (3) acquired vitals. The patient condition trigger can comprise a manual trigger wherein controller 18 determines the NRD of patient P if, for example and without limitation, a caregiver or patient P provides input to user interface 20 that patient P is not feeling well or that the situation of patient P has changed (e.g. patient P is being moved to a different hospital department). The time elapsed since previous measurement trigger can be a setting programmed into controller 18 wherein controller 18 is configured to calculate NRD for patient P at regular intervals. For example and without limitation, controller 18 can be configured to take a NRD measurement at least two hours after the previous NRD measurement and no more than four hours after the previous NRD measurement. The acquired vitals trigger can be a setting programmed into controller 18 wherein controller 18 determines the NRD of patient P if one or more other vital signs being measured by an EWS system or an optional sensor 22 of NRD monitoring system 10 reaches a certain value. For example and without limitation, if one of the optional sensors 22 included in NRD monitoring system 10 is an SpO₂ sensor, the acquired vitals setting can be configured to trigger controller 18 to calculate the NRD index if patient P's SpO₂ drops below a certain level. It will be appreciated that triggers can be activated alone or in varying combinations with one another as deemed appropriate by a caregiver, patient P. or other appropriate party. When any of the trigger conditions are met, patient P is prompted by a specific alarm, e.g. auditory and/or visual, issued through user interface 20 and/or a caregiver, to perform regular breathing and sniff tasks so that controller 18 can calculate the NRD index.

As stated above regarding the vital sign trigger in trigger mode, data sensed by optional non-EMG sensors 22 included in NRD monitoring system 10, such as an accelerometer, a chest SpO2 sensor, or a core temperature sensor, can be used to trigger calculation of the NRD index, but it should be noted that data sensed by optional sensors 22 can also be used to switch controller 18 between a trigger mode and continuous mode. As stated previously, in continuous mode, controller 18 makes an absolute determination of NRD based only on regular breathing EMG signal data without any sniff EMG signal data, and it will be appreciated that when patient P is asleep, operating controller 18 in continuous mode is preferable to operating in trigger mode. However, it will be appreciated that continuous mode can be employed whether patient P is asleep or awake. Either an accelerometer or a core temperature sensor can be used to determine if patient P is asleep. Specifically, accelerometer signal data can be used to determine the posture of patient P. i.e. sitting up or lying down, and it is known that core temperature decreases during sleep and increases during wakefulness.

If accelerometer data indicates that patient P is lying down or a core temperature reading is relatively low, patient P can be presumed to be asleep, especially if patient P also exhibits a low heart rate and/or a low respiration rate. Controller 18 can be configured to automatically switch to operate in continuous mode from trigger mode based on any combination of the factors of sleeping posture, low core temperature, heart rate, and low respiration rate. Conversely, if the accelerometer data indicates that patient P is currently sitting up or moving around more than in the recent past, or if a core temperature reading is relatively high, and if heart rate and/or respiration rate arc relatively high, controller 18 can be configured to automatically switch to operate in trigger mode from continuous mode. Controller 18 is configured to calculate the NRD index based only on regular breathing EMS signal data for the duration of operation in continuous mode.

In another exemplary embodiment, controller 18 can be configured to operate in an enhanced continuous mode, in which controller 18 operates similarly to continuous mode but additionally regularly monitors non-EMG respiratory data in addition to the NRD index. For example and without limitation, in enhanced continuous mode, controller 18 can be configured to measure heart rate, respiration rate and SpO₂ every 30 minutes, in addition to regularly monitoring the NRD index. As with continuous mode, in enhanced continuous mode, controller 18 produces an absolute NRD index based on only regular breathing EMG signal data rather than a relative NRD index based on both regular breathing and sniff EMG data. Accordingly, enhanced continuous mode is, similarly to continuous mode, preferable to trigger mode for a patient P who is sleeping. However, it will be appreciated that enhanced continuous mode can be employed while patient P is awake as well. Both continuous mode and enhanced continuous mode can be referred to collectively as the “continuous modes”.

In an exemplary embodiment, controller 18 can be configured to allow a user (e.g. caregiver or patient P) of NRD monitoring system 10 to manually initiate the calculation of the NRD index at any given time, regardless of whether controller 18 is in trigger mode or one of the continuous modes, and it will be appreciated that the user who manually initiates a NRD calculation can choose whether to initiate a relative NRD calculation requiring patient P to perform a sniff task or an absolute NRD calculation requiring only regular breathing from patient P. In addition, regardless of the trigger mode being differentiated from the continuous modes, it should be noted that, in trigger mode, the controller (18) can be configured to continuously monitor absolute NRD using only regular breathing EMG data between triggered calculations of relative NRD.

As previously stated, the continuous modes can be employed whether patient P is asleep or awake. Although it is generally preferable to employ trigger mode when patient P is awake due to the increased accuracy of a relative NRD index that is based on both regular breathing and sniff EMG data (as demonstrated by ROC curve 30 in FIG. 4), there are situations where it is preferable to use continuous or enhanced continuous mode when patient P is awake. In one non-limiting example, in a hospital general ward, if monitoring of NRD between spot checks by a clinician is desired, and if it is determined that it is not desirable for patient P to sniff without the guidance of a clinician, then continuous or enhanced continuous mode may be employed. Continuous or enhanced continuous mode NRD monitoring may also be desired in order to avoid the “white coat effect”, wherein a patient's vital signs are affected by the stress induced in the patient by being in the presence of a clinician. However, although sniffs may be considered difficult to perform correctly when not overseen by a clinician, controller 18 can be configured to provide appropriate guidance to patient P through user interface 20 for performing a sniff properly if it is desirable to obtain a relative NRD index in the absence of a clinician. However, regardless of whether controller 18 makes a relative or absolute determination of NRD, it is clear that including the NRD monitoring systems and methods disclosed herein in non-invasive EWS systems clearly improves the accuracy of determination of a patient's respiratory status.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A wearable sensor patch for use with a patient monitoring system, the sensor patch comprising:
a number of EMG signal electrodes; and
an EMG reference electrode,
wherein the sensor patch is structured to be affixed to a torso of a patient,
wherein the sensor patch is structured to automatically and non-invasively sense respiratory muscle activity signals used to calculate a neural respiratory drive index, and to automatically and non-invasively sense at least one additional physiological signal other than respiratory muscle activity, and
wherein the sensor patch is configured to be in electrical communication with a power supply and processing means.

2. The sensor patch of claim 1, further comprising:
a number of non-EMG sensors,
wherein the number of non-EMG sensors comprises at least one of: an accelerometer, a SpO₂ sensor, or a core temperature sensor.

3. The sensor patch of claim 1,
wherein the sensor patch is structured such that, when the sensor patch is affixed to the upper torso of the patient, the number of signal electrodes are positioned on the second intercostal space of the patient and the reference electrode is positioned on the sternum of the patient above the reference electrodes.

4. The sensor patch of claim 1, further comprising:
a controller, the controller comprising the power supply and processing means.

5. A patient monitoring system for monitoring a health status of a patient, the monitoring system comprising:
a wearable sensor patch, the sensor patch comprising:
a number of EMG signal electrodes;
an EMG reference electrode; and
an accelerometer;
a controller in electrical communication with the sensor patch; and
a user interface in electrical communication with the controller;
wherein the sensor patch is structured to be affixed to the torso of the patient, wherein the sensor patch is structured to non-invasively sense respiratory muscle activity signals and to sense at least one additional physiological signal other than respiratory muscle activity, and to monitor a plurality of metrics based on the sensed respiratory muscle activity signals and at least one additional physiological signal,
wherein the controller is configured to determine whether or not the patient is in a resting state and whether or not the patient is asleep based on data sensed by the accelerometer and on the plurality of metrics,
wherein the controller is configured to calculate a neural respiratory drive index based on the sensed respiratory muscle activity signals,
wherein the controller is configured to produce a number of health metric scores indicative of a health status of the patient based on the plurality of metrics,
wherein the user interface is configured to display the number of health metric scores.

6. The monitoring system of claim 5,
wherein the sensor patch is structured such that, when the sensor patch is affixed to the upper torso of the patient, the number of signal electrodes are positioned on the second intercostal space of the patient and the reference electrode is positioned on the sternum of the patient above the reference electrodes.

7. The monitoring system of claim 5, wherein the sensor patch
further comprises at least one of a SpO₂ sensor or a core temperature sensor.

8. The monitoring system of claim 5,
wherein the controller is configured to calculate the neural respiratory drive index only if the controller has determined that the patient is at rest.

9. The monitoring system of claim 5,
wherein the controller is configured to be selectively operated in either a trigger mode or in one of a number of continuous modes,
wherein, if the trigger mode has been initiated and any of a number of predetermined trigger conditions are met, the controller is configured to: prompt the patient through the user interface to perform both regular breathing and sniff tasks if the controller has determined that the patient is both resting and awake, and calculate a relative index based on respiratory muscle activity signals sensed during the regular breathing and sniff tasks, and
wherein the controller is configured such that, if any of the number of predetermined trigger conditions are met and the controller has determined that the patient is asleep, the controller will wait until the patient is determined to be awake and resting and then prompt the patient through the user interface to perform both regular breathing and sniff tasks in order to calculate the relative index, and
wherein the controller is configured such that, if any of the number of predetermined trigger conditions are met and the controller has determined that the patient is not resting, the controller will delay calculation of the neural respiratory drive index until the patient is determined to be resting.

10. The monitoring system of claim 9,
wherein, the controller is configured to periodically calculate the absolute index if a continuous mode is initiated, provided that the controller has determined that the patient is resting.

11. A method for monitoring a health status of a patient, the method comprising:
positioning a sensor patch comprising a number of EMG electrodes and a number of non-EMG sensors including an accelerometer on the torso of the patient, the sensor patch being in electrical communication with a controller;
setting the controller to operate in a selected mode of operation, the selected mode of operation comprising either a trigger mode or a continuous mode;
sensing respiratory muscle activity signals and at least one other physiological signal with the sensor patch;
calculating a neural respiratory drive index based on the sensed respiratory muscle activity signals with a controller;
determining a number of health metric scores based on the neural respiratory drive index and the at least one other physiological signal with the controller; and
displaying the number of health metric scores on a user interface in electrical communication with the controller,
wherein the controller is configured to calculate the neural respiratory drive index as either a relative index based on both sniff and regular breathing activity or as an absolute index based only on regular breathing activity.

12. The method of claim 11, wherein setting the controller to operate in the selected mode of operation comprises:
making a determination based on the patient's overall state about whether or not the patient can perform a sniff task on command;
initiating the trigger mode if the initial determination is that a patient can perform a sniff task on command;
choosing to initiate a continuous mode if the initial determination is that a patient cannot perform a sniff task on command;
calculating the relative index in the trigger mode, provided that a number of predetermined trigger conditions is met, that the patient is resting, and that the patient is awake; and
calculating the absolute index in the continuous mode at regular predetermined intervals, provided that the patient is resting.

13. The method of claim 12, further comprising, after initiating trigger mode:
determining with the controller that at least one of the number of predetermined trigger conditions is met;
confirming with the controller whether or not the patient can perform the sniff task on command at the present moment;
instructing the patient to perform relative breathing and sniff tasks if the controller has confirmed that the patient can perform the sniff task on command at the present moment;
calculating the relative index with the controller based on EMG signals sensed by the sensor patch during the regular breathing and sniff tasks;
not calculating the neural respiratory drive index if the controller has not confirmed that the patient can perform the sniff task at the present moment and periodically checking the patient until the controller can confirm that the patient can perform the sniff task at the present moment,
wherein confirming that the patient can perform the sniff task at the present moment comprises determining that the patient is resting and that the patient is awake.

14. The method of claim 12, further comprising, after choosing to initiate a continuous mode:
determining whether a number of enhanced continuous mode trigger conditions should be instated for calculating the neural respiratory drive index;
initiating a standard continuous mode if it is determined that the number of enhanced continuous mode trigger conditions should not be instated;
initiating an enhanced continuous mode if it is determined that the number of enhanced continuous mode trigger conditions should be instated;
calculating the absolute index at regular intervals whether the standard continuous mode or the enhanced continuous mode is initiated; and
provided that the enhanced continuous mode has been initiated, determining with the controller that at least one of the number of enhanced continuous mode trigger conditions is met and calculating the absolute index.

15. The method of claim 14, further comprising:
periodically measuring heart rate, respiration rate, and SpO₂,
wherein the number of non-EMG sensors comprises a chest SpO₂ sensor.
